(19) United States
(12) Patent Application Publication
(10) Pub. No.: US 2018/0310918 A1
(43) Pub. Date: Nov. 1, 2018

(54) Variable focus for shear wave imaging

(71) Applicant: Siemens Medical Solutions USA, Inc., Malvern, PA (US)

(72) Inventors: Liexiang Fan, Sammamish, WA (US); Stephen J. Rosenzweig, Seattle, WA (US)

(21) Appl. No.: 15/498,877

(22) Filed: Apr. 27, 2017

(51) Int. Cl. A61B 8/08

(52) U.S. Cl. CPC A61B 8/485; A61B 8/5253

(57) ABSTRACT

In shear wave imaging with an ultrasound scanner, multiple frames of shear wave data representing the same region of interest are acquired in response to a respective multiple ARFI transmissions. Instead of a fixed or same combination of focal locations for the ARFI transmissions, the focal locations of the ARFI transmissions are varied (e.g., randomly selected) between different frames of shear wave information. By combining the frames, a shear wave image may be generated with less missing data and/or shadowing effects.

### BACKGROUND

The present embodiments relate to shear wave imaging. The shear speed of tissue may be diagnostically useful, so ultrasound is used to estimate the shear speed of a patient's tissue. By transmitting an acoustic radiation force impulse (ARFI), a shear wave is generated at the ARFI focus. Ultrasound scanning monitors the propagation of the shear wave. The arrival time of the shear wave at a distance from the origin of the shear wave is used to determine the velocity of the shear wave in the tissue. The speed for different locations may be estimated, providing a spatial distribution.

Heterogeneous and/or anisotropic tissue may impact shear wave generation, propagation, and detection. Shear wave imaging degrades if the ARFI is applied to areas that do not deform. The nipple, calcification, or other structures may block at least some of the ARFI transmission. Thus, shear wave velocity may not be acquired for some locations. Shadowing or missing of the shear wave information results from the improper application location of the ARFI or detection locations.

### SUMMARY

By way of introduction, the preferred embodiments described below include methods, computer readable storage media with instructions, and systems for shear wave imaging with an ultrasound scanner. Multiple frames of shear wave data representing the same region of interest are acquired in response to a respective multiple ARFI transmissions. Instead of a fixed or same combination of focal locations for the ARFI transmissions, the focal locations of the ARFI transmissions are varied (e.g., randomly selected) between different frames of shear wave information. By combining the frames, a shear wave image may be generated with less missing data and/or shadowing effects.

In a first aspect, a method is provided for shear wave imaging with an ultrasound scanner. A first radiation force pulse is transmitted from a transducer of the ultrasound scanner to a first focus location in a region of interest of tissue of a patient. A first shear wave is generated due to the first radiation force pulse. The ultrasound scanner scans the region of interest with ultrasound as the first shear wave propagates in the region of interest. The scanning providing first data for first locations of the region of interest. A first shear wave characteristic is estimated for each of the first locations from the first data. A second radiation force pulse is transmitted from the transducer of the ultrasound scanner to a second focus location in the region of interest of tissue of the patient. The second focus location is different than the first focus location. A second shear wave is generated due to the second radiation force pulse. The ultrasound scanner scans the region of interest with ultrasound as the second shear wave propagates in the region of interest. The scanning provides second data for the first locations of the region of interest. A second shear wave characteristic is estimated for each of the first locations from the second data. For each of the first locations, the first and second shear wave characteristics are combined. An image of a characteristic of the tissue of the patient is generated from results of the combining.

In a second aspect, a method is provided for shear wave imaging with an ultrasound scanner. Multiple frames of shear wave data responsive to randomly placed focal locations of acoustic radiation force impulses for generating shear waves are acquired. The multiple frames each represent a same region of interest at a different time. The multiple frames are temporally filtered. A shear wave image is generated from the temporally filtered multiple frames.

In a third aspect, a system is provided for shear wave imaging. A transmit beamformer is configured to transmit first and second pushing pulses at first and second, different times to different locations relative to tissue of a patient. A receive beamformer is configured to receive first signals and second signals from scanning after the first and second different times, respectively. An image processor is configured to determine, from the first and second signals, first and second velocities of shear in the tissue, respectively, the first velocities representing locations and second velocities also representing the locations. The image processor is also configured to persist the first velocities with the second velocities. A display is configured to output a shear velocity image from the persisted first and second velocities.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for shear wave imaging with an ultrasound scanner;

FIG. 2 illustrates an example spatial arrangement for ARFI focal locations for shear wave imaging a region of interest;

FIG. 3 shows an example temporal filtering for shear wave imaging; and

FIG. 4 is a block diagram of one embodiment of a system for shear wave imaging.

### DETAILED DESCRIPTION OF THE DRAWINGS AND PRESENTLY PREFERRED EMBODIMENTS

Optimal sensing is provided for shear wave imaging. The radiation force is pseudo-randomly positioned for each frame. The lateral location of each radiation force application is randomly selected in a constrained range, a pseudo-random method. In a time course, the radiation force is applied to different areas of the region of interest. The frames of estimated shear wave characteristics from the different applications are temporally filtered together. The quality of the shear wave source is estimated and is incorporated into the temporal filtering. By using a measure of quality of the shear wave source, the temporal filtering may be weighted to obtain high fidelity. One of or both variation in radiation force applications and shear wave information reconstruction by temporal filtering achieve optimal sensing for a given region of interest.

In one embodiment, the region of the interest is divided into a few sub-regions for each frame of shear wave information. A radiation force application event and a group of pulse echoes events are used to detect the shear wave in each sub-region. The shear waves are detected in all the sub-regions, providing a frame of data for the region of interest. For each frame in the time course of multiple frames, the lateral position of the ARFI application in each sub-region is pseudo-randomly chosen from a limited range. Alternatively, the lateral position of the application is chosen from a pre-defined sequence that varies with each frame. The multiple frames are combined, providing a spatial-temporal combination for the region of interest.

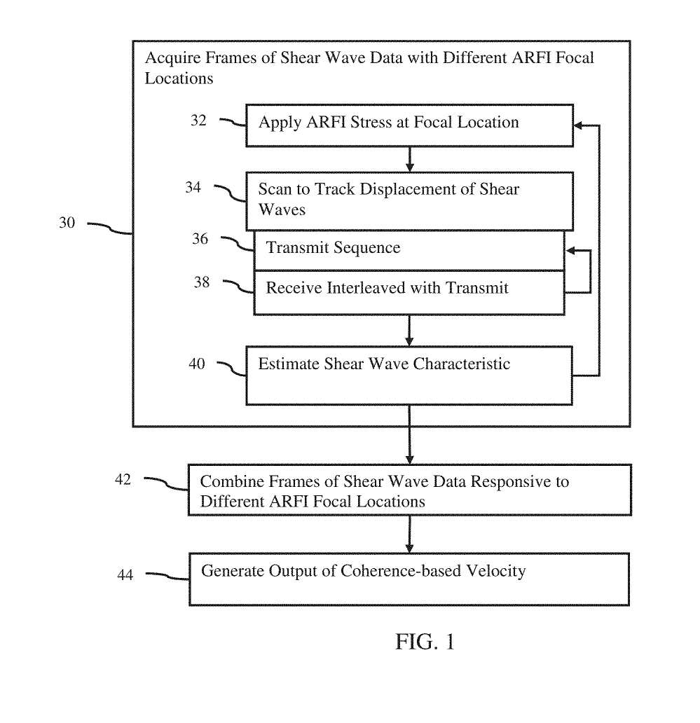

FIG. 1 shows one embodiment of a method for shear wave imaging with an ultrasound scanner. In real-time ARFI imaging, the information of each frame is sampled differently. The sampling of the ARFI focal locations is done intelligently to provide a non-repeated or varied sampling, such as random or pseudo-random. The resulting frames are compounded in the temporal domain.

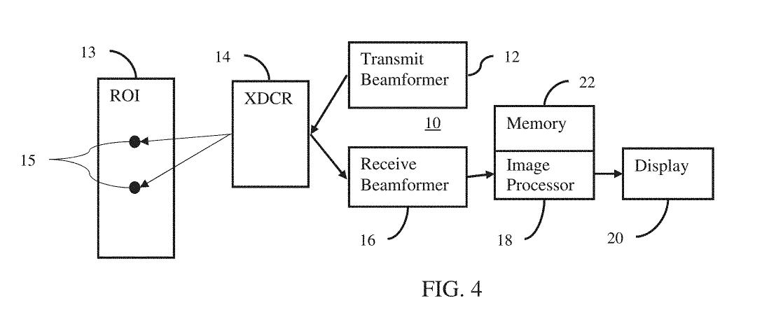

The method is implemented by the system of FIG. 4 or a different system. Transmit and receive beamformers use a transducer to transmit and receive from the patient, including applying ARFI and tracking the tissue response in acts 32-38. An image processor estimates the shear wave characteristic in act 40. The image processor or a filter combines the frames in act 42. The image processor generates the image in act 42. A display may be used for act 46. Different devices, such as other parts of an ultrasound scanner, may perform any of the acts.

The acts are performed in the order described or shown (i.e., top to bottom), but may be performed in other orders. Additional, different, or fewer acts may be provided. For example, acts for configuring the ultrasound scanner, positioning the transducer, identifying a region of interest, and/or recording results are provided. In another example, reference scanning is performed prior to act 32. In alternative embodiments, the initial scan of acts 36 and 38 after generation of the shear waves is used as the reference scan.

To determine tissue motion caused by shear waves, the tissue in a relaxed state or subject to no or relatively little shear wave is detected as a reference. The ultrasound scanner detects reference tissue information. The scanning occurs prior to transmission of the ARFI in act 32, but may be performed at other times. Any type of detection may be used, such as a B-mode detection of the intensity. In other embodiments, the beamformed data without detection is used as the reference.

In act 30, the ultrasound scanner acquires multiple frames of shear wave data. Each frame of shear wave data represents the same locations in the region of interest. All, most, or some of the same locations are represented in each frame. The frames represent the shear wave characteristics of the locations at different times.

Each frame is acquired by transmitting ARFI in act 32 and repetitive scanning in act 34 (e.g., transmitting tracking pulses in act 36 and receiving responsive ultrasound data in act 38). The repetitive scanning tracks displacements of the tissue caused by a shear wave generated from the transmission of act 32. The shear wave characteristic is estimated in act 40 from the ultrasound data.

The focal location of the ARFI and center for tracking the shear wave is at different locations for different frames of data. The variance in location may avoid shadowing or heterogenous tissue effects for some of the frames. By then combining the frames, an image with less shadowing or missing data may result. The different focal locations are in a pattern that does not repeat for the frames to be combined, but may repeat in other embodiments. The different focal locations may be randomly selected from scan locations in the region of interest. In a pseudo-random selection, the focal location or locations for a frame is selected randomly from one of a limited number of options, such as 3-12 options spaced laterally in the region of interest.

In act 32, the ultrasound scanner uses the transducer to apply stress to the tissue. ARFI focused at a point or focal region is transmitted. When ARFI is applied to a focused area, the tissue responds to the applied force by moving. The ARFI creates a shear wave that propagates laterally through the tissue. The shear wave causes displacement of the tissue. At each given spatial location spaced from the focus, this displacement increases and then recovers to zero, resulting in a temporal displacement profile. The tissue properties affect the displacement profile.

The ARFI may be generated by a cyclical pulsed waveform of any number of cycles (e.g., tens or hundreds of cycles). For example, ARFI is transmitted as a pushing pulse with 100-1000 cycles. The transmitted acoustic wave propagates to the region of interest, causing a deposition of energy and inducing a shear wave.

For acquiring different frames of estimates, two or more shear waves are generated. For example, two ARFIs are transmitted from a transducer of the ultrasound scanner at different times. The different ARFIs have some of the same characteristics, such as being at a same center frequency with a same frequency band generated with a same number of cycles, transmit aperture, amplitude, and apodization profile. These characteristics may be different for different ARFIs. Other characteristics may be the same or different.

The ARFIs are transmitted as pushing pulses with different foci. The foci for generating the shear waves are at different locations so that shear waves are generated from different origins, increasing the chance to have fewer or different locations of missing data for one or more frames. In one embodiment, the foci are all at a same depth, but different lateral locations. For tracking displacements, a region of interest is used. This region of interest is set by the user and/or is set based on the spatial distribution of simultaneous receive beams used for tracking. The ARFI foci are at different positions relative to or within the region of interest. The ARFI foci are in and/or outside of the ROI. Any spatial distribution of the foci may be used.

The different focal locations for the different frames are randomly selected. A lateral location is selected from all or a sub-set of lateral locations in or within a given distance of the region of interest. For example, the region of interest is 5 mm across, so the focal location for each frame is randomly selected from one of five options (e.g., every 1 mm of the region of interest) after the region of interest is established. With random selection, the focal locations may be the same for some frames or a check is performed to prevent use of the same focal locations in frames to be combined. In another embodiment, the randomization occurs for programming a predefined sequence of focal locations. In general, this sampling is scheduled in a semi-random fashion or a totally random fashion. For semi-random type of random selection, limitations such as non-repeating or use of only a subset of locations in the region of interest control the options available for random selection.

In yet another embodiment, a non-repeating sequence of different focal locations is predefined and used. The sequence avoids or limits use of the same focal location in frames to be combined.

Any distance between possible focal locations may be used, such as 0.5 mm, 1 mm, at least 2 mm, or other distance. Any number of possible focal locations may be used, such as a number of focal locations based on a number of frames to be combined (e.g., 12 frames to be combined, so 12, 24, or 36 possible focal locations being provided).

The ARFIs or pushing pulses are transmitted at different times. The push pulses are transmitted successively. Any amount of time may separate the transmissions, such as 10 ms. The difference in time is selected so that the shear wave from one pushing pulse attenuates before generating the next shear wave and/or so that tracking of one shear wave is completed before generating the next shear wave. Any interval between ARFI transmissions allows for tracking, transducer cooling, and/or avoiding reaching a limit on applied acoustic energy.

In response to the transmission of the pushing pulses to the different foci in sequence, different shear waves are generated. For example, the shear waves are generated from the different focal locations at the different times in response to the ARFIs. The shear waves travel, in part, towards and/or in the ROI.

In act 34, the ultrasound scanner scans the tissue of the patient. The scanning is repeated any number of times to determine the amount of tissue motion at different locations caused by a shear wave. Acts 36 and 38 provide one embodiment of scanning where a sequence is transmitted and resulting echoes are received. The detected tissue is compared to the reference scan of the tissue over time to determine displacements due to the passing of the shear wave.

Doppler or B-mode scanning may be used for tracking the tissue responding to the stress. Ultrasound data is received in response to transmissions of ultrasound. The transmissions and receptions are performed for different laterally spaced locations, over an area, or over a volume. A sequence of transmissions and receptions are provided for each spatial location to track over time.

Acts 36 and 38 occur after the pushing pulses are applied and while the tissue is responding to the stress. For example, transmission and reception occur after application or change in the stress and before the tissue reaches a relaxed state. Ultrasound imaging is performed before, during and/or after the stress is applied.

In act 36 for tracking, the ultrasound scanner transmits a sequence of transmit beams or tracking pulses. A plurality of ultrasound beams is transmitted to the tissue responding to the stress. The plurality of beams is transmitted in separate transmit events. A transmit event is a contiguous interval where transmissions occur without reception of echoes responsive to the transmission. During the phase of transmitting, there is no receiving. Where a sequence of transmit events is performed, a corresponding sequence of receive events is also performed in act 38 interleaved with the transmissions of act 36. A receive event is performed in response to each transmit event and before the next transmit event.

For a transmit event, one or more transmit beams are formed. Each transmit beam has a frequency response. For example, a transmit beam is formed by a 2.0 MHz pulse of 2 cycles. Any bandwidth may be provided. The pulses to form the transmit beams are of any number of cycles. Any envelope, type of pulse (e.g., unipolar, bipolar, or sinusoidal) or waveform may be used.

In act 38, the transducer receives ultrasound echoes in response to each transmit event. The transducer converts the echoes to receive signals, which are receive beamformed into ultrasound data representing one or more spatial locations. The response of tissue at scan lines for receive beams is detected.

Using reception of multiple receive beams in response to each tracking transmission, data for a plurality of laterally spaced locations may be received simultaneously. The entire ROI is scanned for each receive event by receiving along all the scan lines of the ROI in response to each transmit event. The monitoring is performed for any number of scan lines. For example, four, eight, sixteen, or thirty-two receive beams are formed in response to each transmission. In yet other embodiments, different transmit events and corresponding receive scan lines are scanned in sequence to cover the entire ROI.

The ultrasound scanner receives a sequence of receive signals. The reception is interleaved with the transmission of the sequence. For each transmit event, a receive event occurs. The receive event is a continuous interval for receiving echoes from the depth or depths of interest. After the transducer completes generation of acoustic energy for a given tracking transmission, the transducer is used for reception of the responsive echoes. The transducer is then used to repeat another transmit and receive event pair for the same spatial location or locations, providing the interleaving (e.g., transmit, receive, transmit, receive, . . . ) to track the tissue response over time. The scanning of the region of interest with ultrasound in act 34 is repetitive to acquire ultrasound data representing the tissue response at locations of the region of interest at different times while the shear wave propagates through the region of interest. Each repetition monitors the same region or locations for determining tissue response for those locations. Any number of repetitions may be used, such as repeating about 50-100 times. The repetitions occur as frequently as possible while the tissue recovers from the stress, but without interfering with reception.

In act 40, the ultrasound scanner estimates a shear wave characteristic for each location in the region of interest. The data received by tracking in act 38 is used to detect displacements as a function of time for each location in the region. A maximum or other displacement information over time and/or the locations is used to estimate the shear wave characteristic.

Tissue motion is detected as a displacement in one, two, or three dimensions. Motion responsive to the generated shear waves is detected from the received tracking or ultrasound data output from act 38. By repeating the transmitting of the ultrasound pulses and the receiving of the ultrasound echoes over the time, the displacements over the time are determined. The tissue motion is detected at different times. The different times correspond to the different tracking scans (i.e., transmit and receive event pairs).

Tissue motion is detected by estimating displacement relative to the reference tissue information. For example, the displacement of tissue along scan lines is determined. The displacement may be measured from tissue data, such as B-mode ultrasound data, but flow (e.g., velocity) or beamformer output information prior to detection (e.g., in-phase and quadrature (IQ) data) may be used.

As the tissue being imaged along the scan lines deforms, the B-mode intensity or other ultrasound data may vary. Correlation, cross-correlation, phase shift estimation, minimum sum of absolute differences or other similarity measure is used to determine the displacement between scans (e.g., between the reference and the current scan). For example, each IQ data pair is correlated to its corresponding reference to obtain the displacement. Data representing a plurality of spatial locations is correlated with the reference data. As another example, data from a plurality of spatial locations (e.g., along the scan lines) is correlated as a function of time. For each depth or spatial location, a correlation over a plurality of depths or spatial locations (e.g., kernel of 64 depths with the center depth being the point for which the profile is calculated) is performed. The spatial offset with the highest or sufficient correlation at a given time indicates the amount of displacement. For each location, the displacement as a function of time is determined. Two or three-dimensional displacement in space may be used. One-dimensional displacement along scan lines or along a direction different from the scan lines or beams may be used.

For a given time or repetition of the scanning, the displacements at different locations are determined. The locations are distributed in one, two, or three dimensions. For example, displacements at different laterally spaced locations are determined from averages of displacements of different depths in the ROI. In another example, displacements are determined for different laterally spaced and range spaced (i.e., depth) locations.

In other embodiments, the displacement as a function of location is determined. Different locations have the same or different displacement amplitude. These profiles of displacement as a function of location are determined for different times, such as for each repetition of transmit/receive events in the scanning of act 34. Line fitting or interpolation may be used to determine displacement at other locations and/or other times.

The displacements for one frame of shear data are responsive to the shear wave generated for that frame. Due to the origin location of the shear wave and the relative timing of the scanning for displacement, any given location at any given time may be subject to no shear wave-caused displacement or displacement caused by the shear wave.

The ultrasound scanner calculates the shear wave characteristic for each location from the displacements. Any characteristic may be estimated, such as speed or velocity of the shear wave in the tissue. The shear wave speed of the tissue is a velocity of the shear waves passing through the tissue. Different tissues have different shear wave speed. A same tissue with different elasticity and/or stiffness has different shear wave speed. Other viscoelastic characteristics of tissue may result in different shear wave speed. The shear wave speed is calculated based on the amount of time between the pushing pulse and the time of maximum displacement and based on the distance between the ARFI focal location and the location of the displacements. Other approaches may be used, such as determining relative phasing of the displacement profiles.

Other shear wave characteristics of the tissue may be estimated from the location, displacements, and/or timing. The magnitude of the peak displacement normalized for attenuation, time to reach the peak displacement, Young's modulus, or other elasticity values may be estimated. Any viscoelastic information may be estimated as the shear wave characteristic in the tissue.

Acts 32-40 are repeated for each frame of data representing the shear wave characteristic in the region of interest. Each repetition and corresponding frame provides values for the shear wave characteristic for each location at a different time or period. Values for some locations may be missing due to being the focal location and/or due to missing estimates. For other locations, values are provided for each frame or from frames representing the estimates from different times. The scanning for each repetition may have some locations in common and other locations not in common (i.e., overlapping but not identical fields of locations). The estimation is provided in each repetition for at least some of the locations in common or in the overlapping region.

The estimates for each frame are responsive to a shear wave. The focal locations of the ARFIs for generating the shear waves are different for the different frames of shear wave characteristic. The random, non-repetitive, and/or predefined variation in ARFI focal location for the frames of the sequence results in different sampling of the same locations. Heterogenous tissue may affect estimation for some locations more than others depending on the ARFI focal location. The variation provides some frames with less or more missing data.

Any number of repetitions are used. For example, 5-20 frames of data of shear wave characteristics are generated for combination together. Fewer or more frames may be used. As another example, 2-4 frames per second are acquired. The frames over 2-3 seconds are to be combined.

In one embodiment, the region of interest is separated into two or more sub-regions. For example, a region of interest is 20 mm wide, so is separated into five 4 mm sub-regions (e.g., distinct or non-overlapping sub-regions) or five 5 mm sub-regions (i.e., overlapping sub-regions). Any width may be used. Each sub-region is handled separately. Act 30 is performed for each sub-region. The ARFI transmission, scanning to track tissue, and estimation of shear wave characteristic is performed once for each sub-region, and then repeated across the region of interest. The transmissions of ARFIs for each repetition of act 30 is performed across the sub-regions before each repetition for another frame. This results in a frame of shear wave characteristic data stitched together from the sub-regions. The process is repeated to provide the frames of data over time.

Each sub-region is assigned a plurality of possible locations for the ARFI focal location. Each frame has a same number of ARFI focal locations as there are sub-regions (i.e., one ARFI focal location for each sub-region per frame). For different frames, the ARFI focal locations for the sub-regions are varied. For example, a same number of possible locations and same spatial distribution of possible locations is provided for each sub-region (e.g., sub-region 1 has 5 possible locations from 1-5 mm and sub-region 2 has 5 possible locations from 5 mm-9 mm). One of the possible locations is selected for a given sub-region (e.g., 2 mm). The corresponding possible location is selected for the other sub-regions (e.g., 6 mm). The same relative offset (e.g., 2 mm from left edge) is used for the ARFI focal location for each sub-region. The offset is from an edge, center, or other point of reference. The offset is randomly or semi-randomly selected. For subsequent frames, the selection of ARFI focal locations by selecting the offset for the sub-regions is repeated, providing different ARFI focal locations in each sub-region.

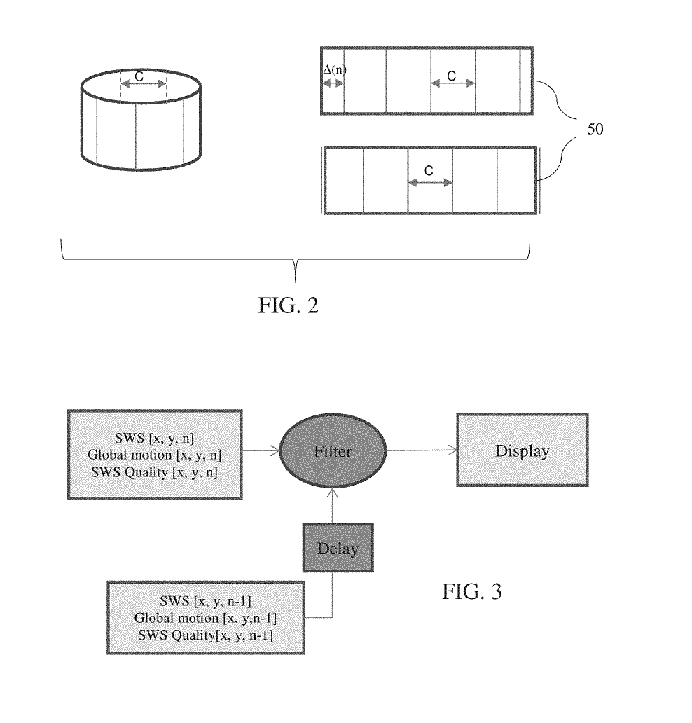

FIG. 2 shows one example. The region of interest 50 is evenly partitioned into a finite number of the small regions of width C. Each region C is a sub-region defining a number of possible ARFI focal locations. The ARFI focal location for each sub-region C is randomly selected. In another approach, the regions C are centered at the selected ARFI location. In concept, the two ends are connected together to represent a closed and limited set of the transmit and receive conditions. A jitter is created with a uniform random distribution [0, C]. This jitter rotates the reference position in the circle. By setting the reference position, this same reference position is used in each sub-section. The right side of FIG. 2 shows the sub-regions C shifted based on random selection for two different frames. The ARFI focal locations are different by Δ(n) between two different frames. After certain number of the frames (n), the reference samples evenly in the C, resulting the highest density of sampling in the spatial domain over time. To generate a full image at a fixed update rate, the full region of interest is sampled at a given temporal interval. The partition of the ARFI beam and the detection region is evenly distributed. From one update to the next, the ARFI beams and the center of the detection region changes based on a random generator.

Referring to act 42 of FIG. 1, a filter or image processor combines the shear wave characteristics from different frames. Each frame provides a value for the shear wave characteristic for each location. Some frames may have missing data for some or all locations. For each location, the values of the shear wave characteristic from the different frames are combined.

The combination is of a set number of frames, such as the frames acquired over a given period. A moving window is used. The frames acquired within a period or a given number of most recently acquire frames are combined. In alternative embodiments, a set number of frames are combined once to generate a single image, unless triggered again. In another embodiment, different combinations are provided over time, such as building up from one frame by combining with each additional frame as acquired.

Any combination may be used. For example, the values are compounded. An average may be calculated. Frames with missing values for that location are not used or not included in the average for that location. The compounding temporally persists the values of the shear wave characteristics. The frames of data are temporally persisted. Any temporal filtering of multiple frames responsive to different ARFI focal locations may be used.

Motion compensation may be provided for the combination. The frames are spatially adjusted relative to each other to account for motion of the tissue and/or transducer occurring between the acquisition of the frames. The motion compensation may be rigid or non-rigid. Any motion compensation may be used, such as determining motion of tissue outside the region of interest using B-mode or speckle tracking. In one embodiment, motion compensation for shear wave imaging is used. Pairs of reference frames are correlated to determine the spatial offset between frames. A polynomial is fit to the spatial offsets to determine a curve of motion over time. In alternative embodiments, motion compensation is not used.

In one embodiment, a weighted combination is used. For example, a weighted average, weighted finite impulse response, or weighted infinite impulse response combination is used. The weight or weights provide a relative weighting of one frame or a previous compound of frames to another frame. The weight may be based on one or more of various factors, such as a number of frames being combined for the location. For example, the weight is a function of qualities of the frames of the shear wave data or quality of the values for the shear wave characteristic at the location. The quality is measured as the signal-to-noise ratio over or of the displacement profile, the signal-to-noise ratio of the beamformed samples (e.g., in-phase and quadrature or radio frequency data), and/or by correlation coefficient between axial and/or azimuth spaced displacement profiles. Frames or values of the shear wave characteristic with greater quality are weighted more heavily in the combination.

FIG. 3 represents one example using frames, n, of shear wave speed (sws) for locations x, y. Two frames, n and n−1, are used, but more frames may be combined. The current and previous frames, sws(n) and sws(n−1), measure of global motion, and sws quality by location are input to the filter. The delay represents use of a previous frame for combination. The quality describes the radiation force as reflected in the data. The global motion is used to align or register the pixels or locations. The global motion is based on the correlation between reference frames. The shear wave quality is used in a weighted mechanism to filter the current frame, sws(n), and the previous frame, sws(n−1), for each aligned spatial location when conducting persistence.

Where the region of interest is divided into sub-regions, the motion compensation and combination may be performed separately for each sub-region. Alternatively, the sub-regions are combined or stitched together with spatial compounding for form the frames. The motion compensation and temporal combination are performed for the full frames.

In act 44 of FIG. 1, the image processor generates an image of a characteristic of the tissue of the patient from results of the combining. The characteristic is the shear wave characteristic. For example, the image is of shear wave velocity in the tissue.

The temporally filtered combination provides values for the shear wave characteristic for each location in the region of interest. The region of interest is user selected or processor determined. Where the ARFI processing is performed by sub-region, then the image is of the combinations of sub-regions to represent the region. The locations are distributed in one, two, or three dimensions. The image is of the shear wave characteristic over the one, two, or three dimensions. For example, a shear wave velocity image is generated from the combination of frames responsive to variation in ARFI focal location.

For each location, the pixel of the image is modulated by the value of the characteristic. Brightness, color, or other modulation may be used. The shear wave image is displayed alone or overlaid on a B-mode or other ultrasound image.

The image may be gradually updated. For example, an initial shear wave image is from a single frame. As the next frame is acquired, the next shear wave image is from a combination of the two frames. As each additional frame is acquired, the frame is added to the combination and the image updated. Once a given number of frames are acquired, a moving window may be used where the frames combined for the image are a most recent number of frames.

In additional or alternative embodiments, the output is a graph or alphanumeric text of the shear wave speed for a location or across locations. The image is of alphanumeric text (e.g., “1.36 m/s”) or overlaid as an annotation on a B-mode or flow-mode image of the tissue. A graph, table, or chart of velocity or velocities may be output as the image.

FIG. 4 shows one embodiment of a system 10 for shear wave imaging. The shear wave images are formed by combining frames of shear wave information responsive to varied placement of the ARFI focus. The system 10 implements the method of FIG. 1 or other methods.

The system 10 is a medical diagnostic ultrasound imaging system or ultrasound scanner. In alternative embodiments, the system 10 is a personal computer, workstation, PACS station, or other arrangement at a same location or distributed over a network for real-time or post acquisition imaging, so may not include the beamformers 12, 16 and transducer 14.

The system 10 includes a transmit beamformer 12, a transducer 14, a receive beamformer 16, an image processor 18, a display 20, and a memory 22. Additional, different or fewer components may be provided. For example, a user input is provided for manual or assisted selection of display maps, selection of tissue properties to be determined, region of interest selection, selection of transmit sequences, or other control.

The transmit beamformer 12 is an ultrasound transmitter, memory, pulser, analog circuit, digital circuit, or combinations thereof. The transmit beamformer 12 is configurable to generate waveforms for a plurality of channels with different or relative amplitudes, delays, and/or phasing. The waveforms are relatively delayed or phased to steer acoustic beams to focal locations. Upon transmission of acoustic waves from the transducer 14 in response to the generated electrical waves, one or more beams are formed. The transmit beams are formed at different energy or amplitude levels. Amplifiers for each channel and/or aperture size control the amplitude of the transmitted beam.

The transmit beamformer 12 is configured to transmit pulses. The transmit beamformer 12 generates ARFI transmissions and tracking transmissions. Different ARFI transmissions are generated at different times. A beamformer controller, the beamformer 12, the image processor 18, and/or a sequence loaded from memory 22 sets the sequence of ARFI beams or pushing pulses. Two or more pushing pulses are transmitted at different times to different locations 15 relative to tissue of interest of the patient. The focal locations 15 are used sequentially where each subsequent focal location 15 occurs after completion of tracking for the previous shear wave before transmission. The locations are in the region of interest 13, but one or more may be outside the region of interest 13.

The different locations are randomly selected, semi-randomly selected, or are selected in a predefined pattern that varies between three, four, five, or more locations (e.g., 12) for the ARFI focus over the frames to be combined. A different focal location may be provided for each frame to be used in a combination. Some locations may be used more than once. The possible focal locations may be evenly or uniformly sampled based on the number of frames to be combined. At least one focal location is provided per frame. Where sub-regions are used, more than one focal location may be provided per frame.

For tracking tissue displacements, a sequence of transmit beams covering the ROI are generated. The sequences of transmit beams are generated to scan a two or three-dimensional region. Sector, vector, linear, or other scan formats may be used. Two or more simultaneous transmit beams may be generated to track the tissue at different locations in the region of interest as the shear wave propagates through the region. The transmit beamformer 12 may generate a plane wave or diverging wave for more rapid scanning.

The ARFI transmit beams may have greater amplitudes than for imaging or detecting tissue motion. Alternatively or additionally, the number of cycles in the ARFI pulse or waveform used is typically greater than the pulse used for tracking (e.g., 100 or more cycles for ARFI and 1-6 cycles for tracking). Aperture differences may be used.

The transducer 14 is a 1-, 1.25-, 1.5-, 1.75-, or 2-dimensional array of piezoelectric or capacitive membrane elements. The transducer 14 includes a plurality of elements for transducing between acoustic and electrical energies. Receive signals are generated in response to ultrasound energy (echoes) impinging on the elements of the transducer. The elements connect with channels of the transmit and receive beamformers 12, 16.

The transmit beamformer 12 and receive beamformer 16 connect with the same elements of the transducer 14 through a transmit/receive switch or multiplexer. The elements are shared for both transmit and receive events. One or more elements may not be shared, such as where the transmit and receive apertures are different (only overlap or use entirely different elements).

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to a transmission. In alternative embodiments, the receive beamformer 16 is a processor for generating samples using Fourier or other transforms. The receive beamformer 16 may include channels for parallel receive beamforming, such as forming two or more receive beams in response to each transmit event. The receive beamformer 16 outputs beam summed data, such as IQ or radio frequency values, for each beam.

The receive beamformer 16 operates during gaps in the sequence of transmit events for tracking. By interleaving receipt of signals with the tracking transmit pulses, a sequence of receive beams are formed in response to the sequence of transmit beams. After each tracking transmit pulse and before the next tracking transmit pulse, the receive beamformer 16 receives signals from acoustic echoes. Dead time during which receive and transmit operations do not occur may be interleaved to allow for reverberation reduction.

The receive beamformer 16 outputs beam summed data representing spatial locations at a given time. Data for different lateral locations (e.g., azimuth spaced sampling locations along different receive scan lines), locations along a line in depth, locations for an area, or locations for a volume are output. Dynamic focusing may be provided. The data may be for different purposes. For example, different scans are performed for B-mode or tissue data than for shear wave velocity estimation. Data received for B-mode or other imaging may be used for estimation of the shear wave velocity. The shear wave at locations spaced from the foci of the pushing pulses are monitored to determine velocity of the shear waves using coherent interference of the shear waves.

The receive beamformer 16 outputs tracking data representing the tissue before, after, and/or during passing of a shear wave. Tracking data is provided to track each sequential shear wave. The tracking data is output for different periods corresponding to the different ARFI transmissions.

The image processor 18 is a B-mode detector, Doppler detector, pulsed wave Doppler detector, correlation processor, Fourier transform processor, application specific integrated circuit, general processor, control processor, image processor, field programmable gate array, digital signal processor, analog circuit, digital circuit, network, server, group of processors, data path, filter, combinations thereof, or other now known or later developed device for detecting and processing information for display from beamformed ultrasound samples. In one embodiment, the image processor 18 includes one or more detectors and a separate processor. The image processor 18 may be one or more devices. Multi-processing, parallel processing, or processing by sequential devices may be used.

The image processor 18 performs any combination of one or more of the acts 40-44 shown in FIG. 1. The image processor 18 may control the transmit and/or receive beamformers 12, 16. Beamformed samples or ultrasound data is received from the receive beamformer 16. The image processor 18 is configured by software, hardware, and/or firmware.

The image processor 18 is configured to detect displacements of tissue responding to acoustic radiation force. The detection is from beamformed samples or detected data (e.g., B-mode or Doppler detection) from the beamformed samples. Using correlation, other measure of similarity, or another technique, the movement of tissue relative to a reference is determined from the ultrasound data. By spatially offsetting a tracking set of data relative to a reference set of data in one, two, or three-dimensional space, the offset with the greatest similarity indicates the displacement of the tissue. The processor 18 detects displacement for each time and location. Some of the detected displacements may have magnitudes responsive to a passing shear wave or shear waves.

The image processor 18 is configured to determine a velocity or other shear wave characteristic of shear in the tissue. The determination is based on the signals from tracking the tissue responding to the shear waves created by an ARFI. The signals are used to detect the displacements. To determine the velocity, the displacements are used. The time to reach a maximum displacement and distance from the ARFI focal location provide the velocity. Relative phasing of displacements over time of different locations or other approaches may be used to determine velocity.

For each ARFI or the ARFIs used to cover the entire region of interest once, the image processor 18 determines velocities or another characteristic. Frames of data of the characteristic are generated. The frames represent the shear wave interaction with the tissue at different times in response to different ARFI focal locations. For example, frames of velocity representing the same locations and responsive to different ARFI focal locations are generated.

The image processor 18 is configured to persist the velocities from the different frames. Any number of frames are combined. A moving window indicating the frames to combine may be used. Since the frames represent the tissue response to shear at different times, temporal filtering is used. For each location, the velocities or other characteristic are averaged, weighted averaged, or combined in some way.

The image processor 18 may vary the contribution of a given frame to the persistence. The variation is by frame (e.g., values of entire frame weighted more or less heavily than for other frames) or by location (e.g., value for one frame at one location more heavily weighted than value for that same frame at a different location). Any measure may be used to vary the contribution, such as time (e.g., older frames weighted less). In one embodiment, the quality of the characteristic is used. The contribution to the persistence is weighted based on the relative quality of the data being combined.

The image processor 18 generates display data, such as annotation, graphic overlay, and/or image. The display data is in any format, such as values before mapping, gray scale or color-mapped values, red-green-blue (RGB) values, scan format data, display or Cartesian coordinate format data, or other data. The processor 18 outputs velocity information appropriate for the display device 20, configuring the display device 20. Outputs to other devices may be used, such as outputting to the memory 22 for storage, output to another memory (e.g., patient medical record database), and/or transfer over a network to another device (e.g., a user computer or server).

The display device 20 is a CRT, LCD, projector, plasma, printer, or other display for displaying shear velocity, graphics, user interface, validation indication, two-dimensional images, or three-dimensional representations. The display device 20 displays ultrasound images, the velocity, and/or other information. For example, the display 20 outputs tissue response information, such as a one, two, or three-dimensional distribution of the velocity or other shear wave characteristic. Velocities or shear wave characteristics for different spatial locations form an image. The output of the persistence or combination of characteristics from different frames with different ARFI focal locations is used for imaging. The combination from variably placed focal locations reduces missing data and/or shadowing in the shear wave imaging. Other images may be output as well, such as overlaying the velocity as a color-coded modulation for a region of interest on a gray scale B-mode image.

In one embodiment, the display device 20 outputs an image of a region of the patient, such as a two-dimensional Doppler tissue or B-mode image. The image includes a location indicator for the velocity. The location indicator designates the imaged tissue for which a velocity value is calculated. The velocity is provided as an alphanumeric value on or adjacent the image of the region. The image may be of the alphanumeric value with or without spatial representation of the patient.

The processor 18 operates pursuant to instructions stored in the memory 22 or another memory. The memory 22 is a computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The memory 22 alternatively or additionally stores data used in estimation of shear wave characteristic using variable ARFI focal locations and compounding. For example, the transmit sequences and/or beamformer parameters for ARFI and tracking are stored. As another example, the region of interest, received signals, detected displacements, estimated shear wave characteristic values, filter or persistence settings, weights, quality measures, filter outputs, and/or display values are stored.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for shear wave imaging with an ultrasound scanner, the method comprising:
transmitting a first radiation force pulse from a transducer of the ultrasound scanner to a first focus location in a region of interest of tissue of a patient, a first shear wave being generated due to the first radiation force pulse;
scanning, by the ultrasound scanner, the region of interest with ultrasound as the first shear wave propagates in the region of interest, the scanning providing first data for first locations of the region of interest;
estimating a first shear wave characteristic for each of the first locations from the first data;
transmitting a second radiation force pulse from the transducer of the ultrasound scanner to a second focus location in the region of interest of tissue of the patient, the second focus location different than the first focus location, a second shear wave being generated due to the second radiation force pulse;
scanning, by the ultrasound scanner, the region of interest with ultrasound as the second shear wave propagates in the region of interest, the scanning providing second data for the first locations of the region of interest;
estimating a second shear wave characteristic for each of the first locations from the second data;
combining, for each of the first locations, the first and second shear wave characteristics; and
generating an image of a characteristic of the tissue of the patient from results of the combining.

2. The method of claim 1 wherein transmitting the first and second radiation force pulses comprises transmitting with the first and second focus locations being randomly selected in the region of interest.

3. The method of claim 1 wherein transmitting the first and second radiation force pulses comprises transmitting with the first and second focus locations offset laterally by at least 2 mm.

4. The method of claim 1 wherein transmitting the first and second radiation force pulses comprises transmitting with the first and second focus locations offset in a predefined sequence for each frame of the shear wave characteristic for the region of interest.

5. The method of claim 1 wherein scanning comprises repetitively transmitting tracking pulses over the region of interest and receiving acoustic responses responsive to the tracking pulses.

6. The method of claim 1 wherein estimating the first and second shear wave characteristics comprises detecting displacements as a function of time for the first locations and finding a maximum displacement from the displacements as a function of time for each of the first locations.

7. The method of claim 1 wherein estimating the first and second shear wave characteristic comprises estimating shear wave velocity.

8. The method of claim 1 wherein generating the image comprises generating the image as a multi-dimensional spatial representation of the characteristic.

9. The method of claim 1 wherein generating the image comprises generating a shear wave image.

10. The method of claim 1 wherein combining comprises weighted combination with weights being a function of a measure of quality of the first and second shear wave characteristics.

11. The method of claim 1 wherein combining comprises temporally persisting.

12. The method of claim 1 wherein the first and second shear wave characteristic are the characteristic of the image, further comprising repeating the transmitting, scanning, and estimating, each repetition providing a frame of data for the characteristic, and wherein combining comprises temporally filtering the frames of data.

13. The method of claim 1 further comprising separating the region of interest into two or more sub-regions, wherein the first and second focal locations are in a first of the sub-regions, further comprising repeating the transmitting to multiple, different focal locations, scanning, and estimating for each of the other sub-regions, wherein combining comprises combining for each sub-region, and wherein generating the image comprises generating the image of the region of interest from the combinations for each sub-region.

14. The method of claim 13 wherein the focal locations for each sub-region, including the first and second focal locations of the first sub-region, are at a same relative offset from a center of the respective sub-region for each repetition, the relative offset being randomly selected for each repetition, and wherein the transmitting for each repetition is performed across the sub-regions before each repetition.

15. A method for shear wave imaging with an ultrasound scanner, the method comprising:
acquiring multiple frames of shear wave data responsive to randomly placed focal locations of acoustic radiation force impulses for generating shear waves, the multiple frames each representing a same region of interest at a different time;
temporally filtering the multiple frames; and
generating a shear wave image from the temporally filtered multiple frames.

16. The method of claim 15 wherein acquiring comprises transmitting the acoustic radiation force impulses focused at the randomly placed focal locations in the region of interest, tracking displacements of tissue resulting from the shear waves, and estimating shear wave velocity from the displacements.

17. The method of claim 15 wherein temporally filtering comprises a weighted combination of the frames representing locations in the region of interest with weights of the weighted combination being a function of qualities of the frames of the shear wave data.

18. The method of claim 15 wherein generating the shear wave image comprises generating a shear velocity image of the region of interest.

19. A system for shear wave imaging, the system comprising:
a transmit beamformer configured to transmit first and second pushing pulses at first and second, different times to different locations relative to tissue of a patient;
a receive beamformer configured to receive first signals and second signals from scanning after the first and second different times, respectively;
an image processor configured to determine, from the first and second signals, first and second velocities of shear in the tissue, respectively, the first velocities representing locations and second velocities also representing the locations, and configured to persist the first velocities with the second velocities; and
a display configured to output a shear velocity image from the persisted first and second velocities.

20. The system of claim 19 wherein the transmit beamformer is configured to transmit the first and second pushing pulses to focal positions randomly chosen in a region of interest, wherein the image processor is configured to persist as a function of quality of the first and second velocities, and wherein the shear velocity image is a spatial distribution of shear velocity in the region of interest.
